(19) United States
(12) Patent Application Publication
(10) Pub. No.: US 2011/0054593 A1
(43) Pub. Date: Mar. 3, 2011

(54) SHEATHLESS EMBOLIC PROTECTION DEVICE

(75) Inventors: Erin Guest, Minneapolis, MN (US); Benjamin Arcand, Minneapolis, MN (US); James Anderson, Fridley, MN (US)

(73) Assignee: BOSTON SCIENTIFIC SCIMED, INC., Maple Grove, MN (US)

(21) Appl. No.: 12/549,609

(22) Filed: Aug. 28, 2009

(51) Int. Cl. A61F 2/06

(52) U.S. Cl. 623/1.23

(57) ABSTRACT

An intravascular distal protection device, comprising a guidewire having a proximal end and a distal end, a filtering material disposed about the guidewire, the filtering material having a proximal edge defining a proximally facing opening when in an expanded position, a hoop wire having a proximal end and a distal end and slidably attached to the proximal edge, the hoop wire wrapped in a helical coil about the guidewire in a first contracted position and defining a hoop at the proximal end of the filtering material in the expanded position, and an actuation member attached to the hoop wire and extending at least substantially to the proximal end of the guidewire and configured to move the hoop wire between the first contracted position and the expanded position.

### FIELD OF THE INVENTION

The invention pertains to the field of percutaneous filters such as intravascular filters and more particularly to expandable or actuatable filters and methods of use thereof.

### BACKGROUND

Heart disease is a major problem in the United States and throughout the world. Conditions such as atherosclerosis result in blood vessels becoming blocked or narrowed. This blockage can result in too little oxygen getting to the heart, which has significant consequences as the heart muscle must be well oxygenated to maintain its blood pumping function.

Occluded, stenotic or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over the guidewire such that the balloon is positioned adjacent a stenotic lesion. The balloon is then inflated to open the restriction of the blood vessel. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions such as the neural or the pulmonary vasculature and lead to undesirable results. During angioplasty procedures, stenotic debris may also break loose due to manipulation of the blood vessel. Because of this debris, a number of devices, termed distal protection devices, have been developed to filter out this debris or prevent the debris from moving downstream from the procedure site. There is an ongoing need for new and improved distal protection devices.

### SUMMARY

One embodiment pertains to an intravascular distal protection device, comprising a guidewire having a proximal end and a distal end, a filtering material disposed about the guidewire, the filtering material having a proximal edge defining a proximally facing opening when in an expanded position, a hoop wire having a proximal end and a distal end and slidably attached to the proximal edge, the hoop wire wrapped in a helical coil about the guidewire in a first contracted position and defining a hoop at the proximal end of the filtering material in the expanded position; and an actuation member attached to the hoop wire and extending at least substantially to the proximal end of the guidewire and configured to move the hoop wire between the first contracted position and the expanded position. In some embodiments, the actuation member is attached to the distal end of the hoop wire. In some embodiments, the actuation member is attached to the proximal end of the hoop wire. The helical coil includes two or more turns around the guidewire. The guidewire may be hollow along at least a portion and wherein the actuation member is at least partially disposed within the guidewire. The proximal edge of the filtering material may include a hem disposed on the hoop wire or may include a plurality of attachment rings disposed on the hoop wire. One or more coils may be disposed between the attachment rings on the hoop wire. A stop mechanism may connect the ends of the hoop when in the expanded position, where the stop mechanism includes a ring slidably disposed around the guidewire and connected to one of the hoop wire ends. The hoop wire may be hollow, and the actuation member may be disposed within the hoop wire such that tension in the actuation member brings the proximal and distal ends of the hoop wire together.

Another embodiment pertains to an intravascular distal protection device, comprising a hollow guidewire having a proximal end and a distal end, a filtering material having a proximal edge defining a proximally facing opening when in an expanded position, a hoop wire having a proximal end and a distal end and slidably attached to the proximal edge, the hoop wire having a reduced profile in a first contracted position and defining a hoop at the proximal end of the filtering material in the expanded position; and an actuation member attached to the distal end of the hoop wire and configured to move the hoop wire between the first contracted position and the expanded position, the actuation member disposed within the guidewire. The hoop wire may be wrapped about the actuation member in the contracted position. The filtering material may preferably be at least partially distal the distal end of the guidewire. The proximal edge of the filtering material may include a hem disposed on the hoop wire or a plurality of attachment rings disposed on the hoop wire with one or more coils disposed between the attachment rings on the hoop wire.

Another embodiment pertains to a method of using the device of claim 1, comprising the steps of positioning a delivery catheter proximal of an area of interest, the delivery catheter having an open distal end, moving the device distally through the distal end of the delivery catheter and across the area of interest while the hoop wire is in the first contracted position, and deploying the filtering material into the expanded position by moving the actuation member relative to the guidewire, wherein the step of deploying the filtering material may include the step of pulling on the actuation member.

The above summary of some embodiments is not intended to describe each disclosed embodiment or every implementation of the present invention. The Figures and Detailed Description that follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a diagrammatic side view of a first embodiment of a distal protection device with the device in an expanded configuration.

FIG. 2 is a diagrammatic side view of the embodiment of FIG. 1 with the distal protection device in a collapsed configuration.

FIG. 3 is a diagrammatic side view of a second embodiment of a distal protection device with the device in an expanded configuration.

FIG. 4 is a diagrammatic side view of the embodiment of FIG. 3 with the distal protection device in a collapsed configuration.

FIG. 5 is a diagrammatic perspective view of another embodiment of a distal protection device in an expanded configuration.

FIG. 6 is a diagrammatic side view of another embodiment of a distal protection device with the device in an expanded configuration.

FIG. 7 is a diagrammatic side view of the embodiment of FIG. 6 with the device in a collapsed configuration.

FIG. 8 is an exemplary diagrammatic view of a portion of a distal protection device.

FIG. 9 is a diagrammatic side view of another embodiment of a distal protection device with the device in an expanded configuration.

FIG. 10 is a diagrammatic side view of the embodiment of FIG. 9 with the device in a collapsed configuration.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

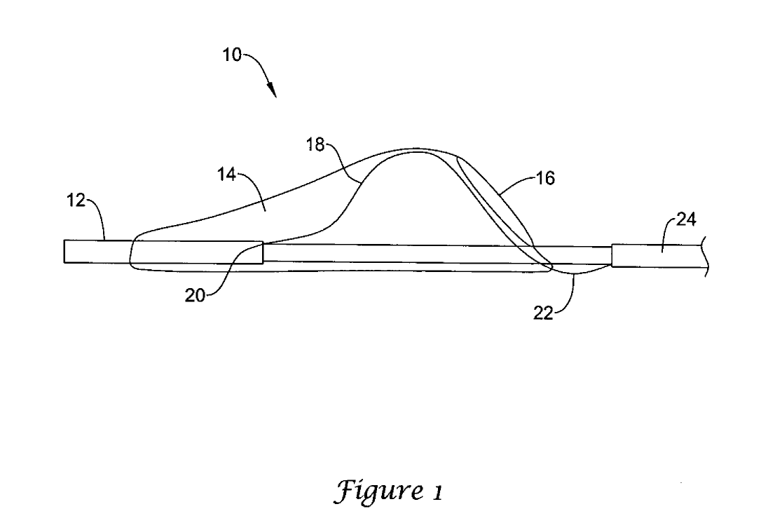

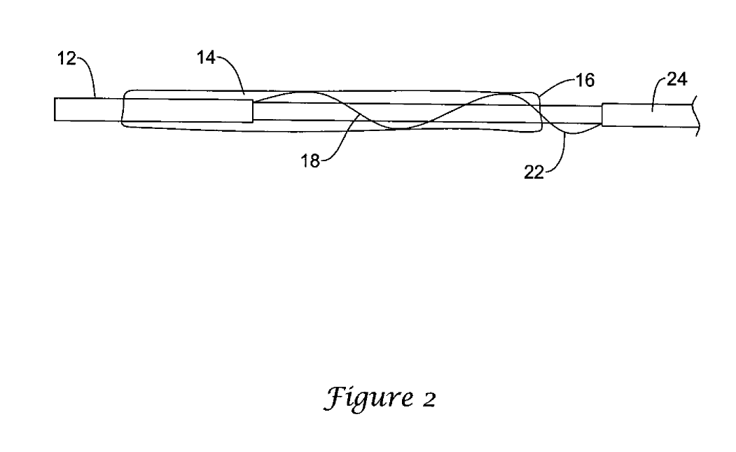

Turning now to FIGS. 1 and 2, which are diagrammatic side views of the distal portion of a first embodiment of a distal protection device with the device in an expanded configuration and a collapsed configuration, respectively, embolic distal protection device 10 includes a guidewire 12 with a filtering membrane 14 disposed around the guidewire. The guidewire may include an atraumatic distal tip at the distal end. Filtering membrane 14 defines a cavity with a proximally facing mouth defined by a proximal edge 16 of the filtering membrane when in the expanded position. A hoop wire 18 is disposed in the filtering membrane 14 and is slidably affixed to the proximal edge 16 of the filtering membrane 14. A distal end 20 of the hoop wire may be fixed to guidewire 12 and and a proximal end 22 of the hoop wire may be fixed to an actuation member 24. In this embodiment, actuation member 24 is a tubular member slidably disposed about the guidewire 12.

The distal protection device is preferably actuated from the contracted position illustrated in FIG. 2 to the expanded position illustrated in FIG. 1. In the contracted position, the hoop wire 18 is coiled about the guidewire as shown in FIG. 2. The coil is a generally helical coil with the loops spaced apart. There may be one, two, three or more loops of the coil depending on the particular embodiment. In the embodiment shown, the hoop wire is coiled into approximately two loops. Because the proximal edge of the filtering membrane 14 is slidably disposed on the hoop wire, the filtering membrane is drawn towards the guidewire and is preferably twisted about the guidewire. In this manner, a low profile contracted position is obtained without the need for a delivery sheath, cardigan or other restraining mechanism. The distal protection device 10 is actuated to the expanded position of FIG. 1 by moving the actuation device distally with respect to the guidewire. This allows the hoop wire to expand into a hoop shape to give the mouth a circular shape. In some embodiments, the hoop wire is biased to expand into the hoop shape. The hoop wire slides through the proximal edge 16 of the filtering membrane and forms the proximal edge into a mouth ready to receive and trap emboli. In some embodiments, the hoop wire is held in place in the contracted position primarily by tension placed on the hoop wire by the actuation member without significant coiling of the actuation member about the guidewire. In other embodiments, the hoop wire is help in place in the contracted position by coiling it about the guidewire as described above.

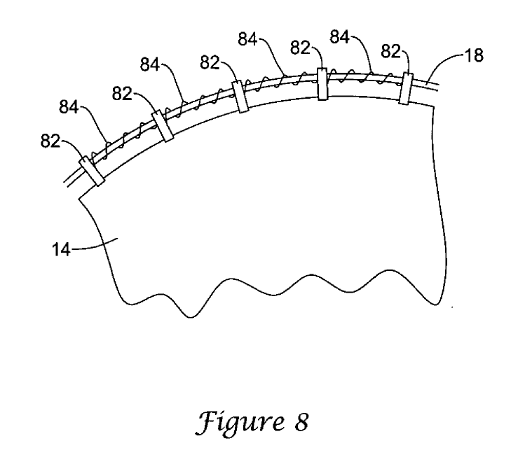

The hoop wire is slidably disposed within the proximal edge of the filtering membrane. In some embodiments, the filtering membrane may be formed into a hem around the filtering member. In some embodiments as shown in FIG. 8, which is a partial diagrammatic view of a hoop wire 18 and filtering membrane 14, attachments rings 82 may attach the proximal edge of the filtering membrane to the hoop wire. In these embodiments, there may be one or more coils 84 disposed between the attachment rings that are compressed when the distal protection device is in the contracted position and expand when the distal protection device is moved to the expanded position to help the filtering membrane open. These coils may be radiopaque.

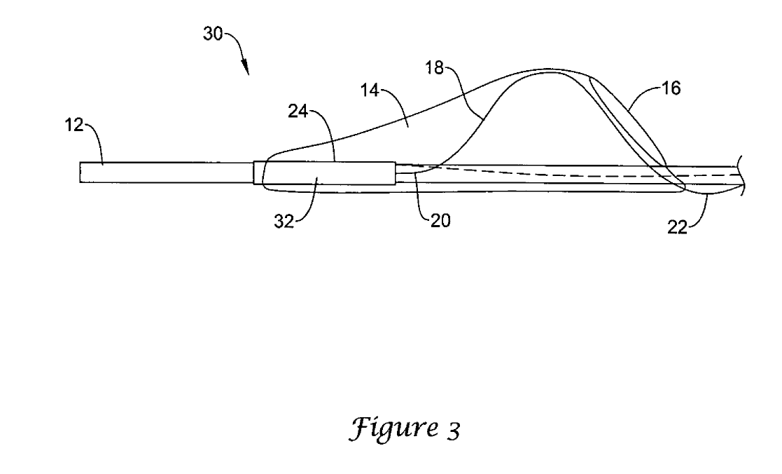

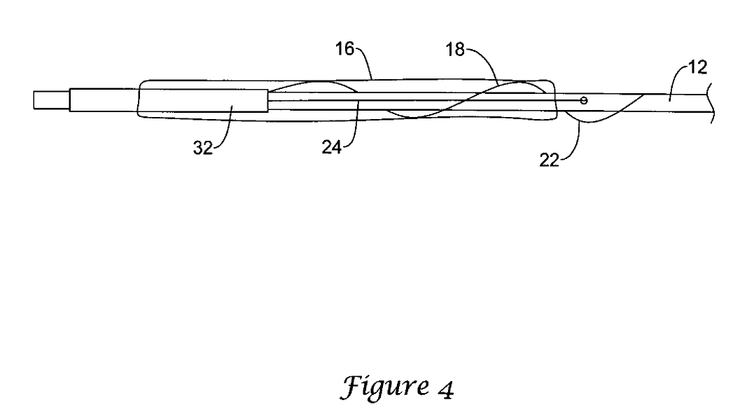

FIGS. 3 and 4 are diagrammatic side views of a second embodiment of a distal protection device with the device in an expanded configuration and in a collapsed configuration, respectively. A distal protection device 30 includes a guidewire 12 with a filtering membrane 14 disposed around the guidewire. The guidewire may include an atraumatic distal tip at the distal end. Filtering membrane 14 defines a cavity with a proximally facing mouth defined by a proximal edge 16 of the filtering membrane when in the expanded position. A hoop wire 18 is disposed in the filtering membrane 14 and is slidably affixed to the proximal edge 16 of the filtering membrane 14. A distal end 20 of the hoop wire may be fixed to an actuation member 24 and a proximal end 22 of the hoop wire may be fixed to guidewire 12. In this embodiment, actuation member 24 is a tubular member slidably disposed within a hollow guidewire 12. A stop mechanism 32 may be included, which stop mechanism may be a tubular member disposed about the guidewire and which, when the distal protection device is in the expanded position, positions the distal end of the hoop wire near the proximal end to reconnect the hoop.

The embolic protection device may be positioned in the contracted position as shown in FIG. 4 with the hoop wire 18 coiled about the guidewire. This has the effect of pulling the filtering member into a low profile about the guidewire for delivery without a sheath or other additional restraining member. The actuating member is pulled proximally relative to the guidewire to move the distal protection device into the expanded configuration. The hoop wire expands, opening up the filtering membrane and forming a proximal mouth for receiving and trapping embolic material.

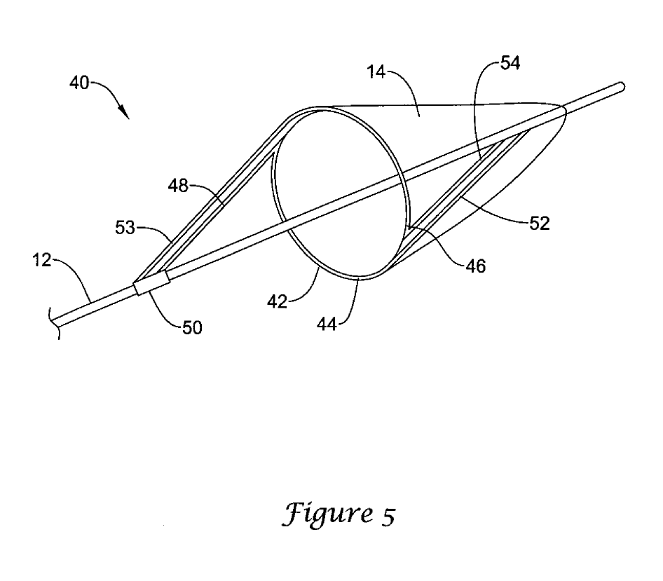

FIG. 5 is a diagrammatic perspective view of another embodiment of a distal protection device in an expanded configuration. Distal protection device 40 includes a guidewire 12 with a filtering membrane 14 disposed about the guidewire. A hoop 42 includes a first hoop arc 44 and a second hoop arc 46. First hoop arc 44 is attached by a proximal strut 48 to a ring 50 and by a distal strut 52 to guidewire 12. Second hoop arc 46 is likewise attached to ring 50 by a proximal strut 52 and to guidewire 12 by a distal strut 54. In an expanded configuration, as shown, the two hoop arcs expand to form the mouth of the hoop and the hoop arcs are slidably affixed to the proximal edge of the filtering membrane in a manner as discussed above. Ring 50 is slideably disposed on guidewire 12 and may be pulled distally by an actuation member (not shown) to collapse the filtering membrane for delivery.

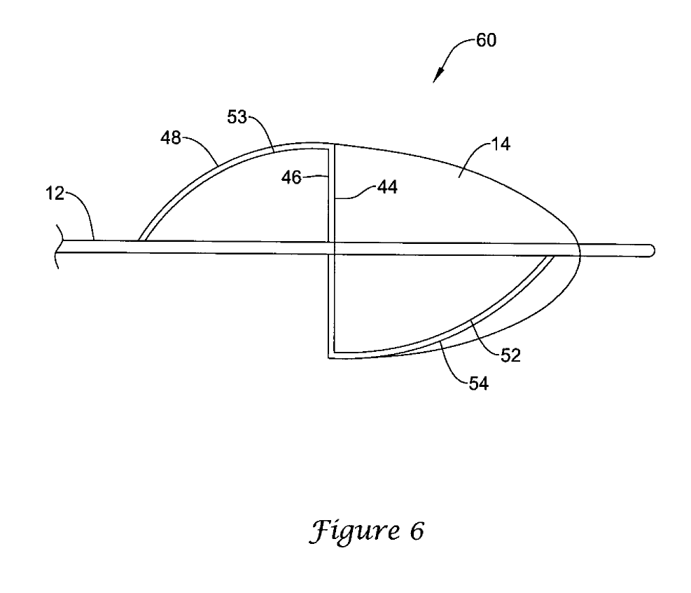

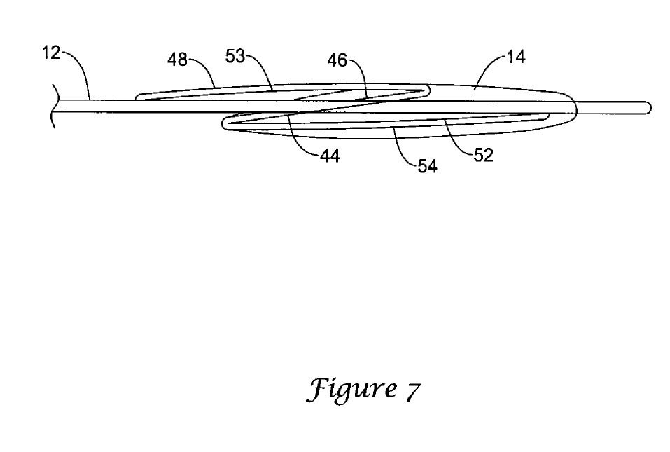

FIGS. 6 and 7 are diagrammatic side views of another embodiment of a distal protection device with the device in an expanded configuration and in a collapsed configuration, respectively. Distal protection device 60 has first and second hoop arcs 44 and 46 as described above. In this embodiment, proximal struts 48 and 52 are curved as shown and are attached fixedly to the guidewire. Distal struts 46 and 54 are similarly arced. For the collapsed configuration, hoop arcs 44 and 46 are rotated down and made to lie straight against the guidewire 12. This also straightens out and forces the struts to lie flat against the guidewire 12 as well. The struts and the filtering membrane 14 may be retained in this position by a delivery sheath, cardigan or similar device, which may be removed when deployment is desired.

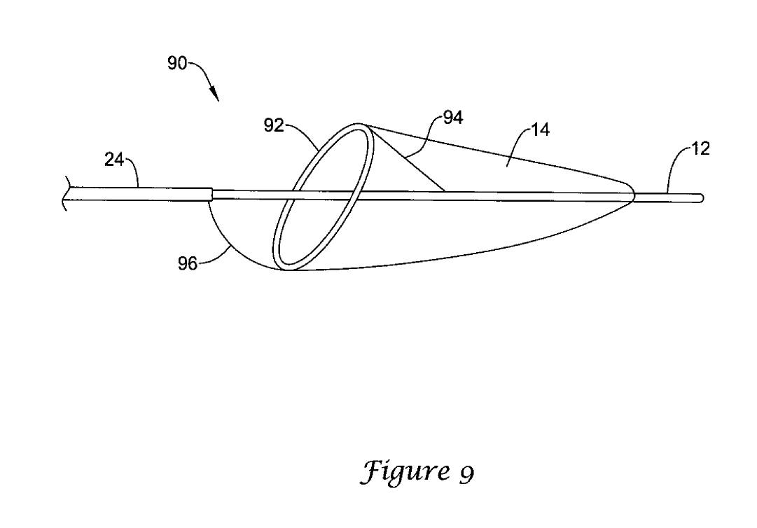

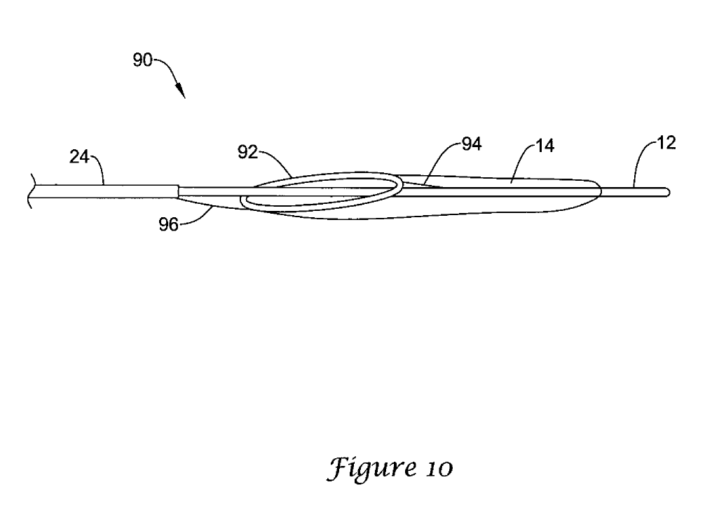

FIGS. 9 and 10 are diagrammatic side views of another embodiment of a distal protection device with the device in an expanded configuration and in a collapsed configuration, respectively. This distal protection device 90 includes a guidewire 12 and a filtering membrane 14. A hoop 92 is fixed to the proximal edge 16 of the filtering membrane 14. A distal strut 94 extends distally from the hoop 92 to fix to guidewire 12. A proximal strut 96 extends proximally from the filter mouth to attach to an actuation member 24. The actuation member may be, as is shown, a tube slidably disposed about the guidewire 12 or another suitable element. In use, this embodiment is collapsed by pulling proximally on the actuation member, which forces the struts and the hoop flat against the guidewire.

Below, some embodiments are described through the use of claim phraseology:

1. An intravascular distal protection device, comprising:

a guidewire having a proximal end and a distal end;

a filtering material disposed about the guidewire, the filtering material having a proximal edge defining a proximally facing opening when in an expanded position;

a hoop wire having a proximal end and a distal end and slidably attached to the proximal edge, the hoop wire wrapped in a helical coil about the guidewire in a first contracted position and defining a hoop at the proximal end of the filtering material in the expanded position; and

an actuation member attached to the hoop wire and extending at least substantially to the proximal end of the guidewire and configured to move the hoop wire between the first contracted position and the expanded position.

2. The device of embodiment 1, wherein the actuation member is attached to the distal end of the hoop wire.

3. The device of embodiment 1, wherein the actuation member is attached to the proximal end of the hoop wire.

4. The device of any of the preceding embodiments, wherein the helical coil includes two or more turns around the guidewire.

5. The device of any of the preceding embodiments, wherein the guidewire is hollow along at least a portion and wherein the actuation member is at least partially disposed within the guidewire.

6. The device of any of the preceding embodiments, wherein the proximal edge of the filtering material includes a hem disposed on the hoop wire.

7. The device of any of embodiments 1-5, wherein the proximal edge of the filtering material includes a plurality of attachment rings disposed on the hoop wire.

8. The device of embodiment 7, wherein the proximal edge of the filtering material includes one or more coils disposed between the attachment rings on the hoop wire.

9. The device of any of the preceding embodiments, wherein the filter has a length, and wherein in the first contracted position, the proximal end of the hoop wire is spaced apart from the distal end of the hoop wire a distance that is at least half the length of the filter.

10. The device of any of the preceding embodiments, wherein the distance is at least three-quarters the length of the filter.

11. The device of any of the preceding embodiments, further comprising a stop mechanism to connect the ends of the hoop when in the expanded position, the stop mechanism including a ring slidably disposed around the guidewire and connected to one of the hoop wire ends.

12. The device of any of the preceding embodiments wherein the hoop wire is hollow, wherein the actuation member is disposed within the hoop wire and wherein tension in the actuation member brings the proximal and distal ends of the hoop wire together.

13. An intravascular distal protection device, comprising:

a hollow guidewire having a proximal end and a distal end;

a filtering material having a proximal edge defining a proximally facing opening when in an expanded position;

a hoop wire having a proximal end and a distal end and slidably attached to the proximal edge, the hoop wire having a reduced profile in a first contracted position and defining a hoop at the proximal end of the filtering material in the expanded position; and

an actuation member attached to the distal end of the hoop wire and configured to move the hoop wire between the first contracted position and the expanded position, the actuation member disposed within the guidewire.

14. The device of embodiment 13 wherein the hoop wire is wrapped about the actuation member in the contracted position.

15. The device of embodiment 13 or 14 wherein the filtering material is at least partially distal the distal end of the guidewire.

16. The device of embodiments 13-15, wherein the proximal edge of the filtering material includes a hem disposed on the hoop wire.

17. The device of embodiments 13-15, wherein the proximal edge of the filtering material includes a plurality of attachment rings disposed on the hoop wire.

18. The device of embodiment 17, wherein the proximal edge of the filtering material includes one or more coils disposed between the attachment rings on the hoop wire.

19. A method of using the device of any of the preceding embodiments, comprising the steps of:

positioning a delivery catheter proximal of an area of interest, the delivery catheter having an open distal end;

moving the device distally through the distal end of the delivery catheter and across the area of interest while the hoop wire is in the first contracted position; and

deploying the filtering material into the expanded position by moving the actuation member relative to the guidewire.

20. The method of embodiment 20, wherein the step of deploying the filtering material includes the step of pulling on the actuation member.

21. A distal protection device, comprising:

a guidewire having a proximal end and a distal end;

a filtering material forming, in an expanded configuration, a filter defining a cavity having a proximally facing mouth;

a hoop affixed to the filter mouth;

a first strut attached to the hoop and extending distally from the hoop, the first strut having a distal end affixed to the guidewire.

A second strut attached to the hoop and extending proximally from the hoop, the second strut having a proximal end affixed to the guidewire.

22. The device of embodiment 21, wherein the first and second struts are rotatably affixed to the guidewire.

23. The device of embodiment 21, wherein the second strut is slidably affixed to the guidewire and further comprising an actuation member configured to move the proximal end of the second strut proximally and distally.

24. The device of embodiment 23, wherein the second strut is attached to a ring slidably disposed on the guidewire.

25. The device of embodiment 23,

wherein the hoop comprises a first hoop arc and a second hoop arc, the first and second hoop arcs together substantially extending around the perimeter of the filter mouth,

wherein the first hoop arc is fixed to the first and second struts, and

further comprising a third and fourth strut, wherein the third strut is fixed to the second hoop arc and extends distally therefrom, the third strut having a distal end attached to the guidewire, wherein the fourth strut is fixed to the second hoop arc and extends proximally therefrom and has a proximal end slidably affixed to the guidewire.

26. The device of embodiment 25 wherein the first hoop arc has a first end and a second end and wherein the first strut is attached to the first end and the second strut is attached to the second end, and wherein the second hoop arc has a first end and a second end and wherein the third strut is attached to the first end of the second hoop arc and the fourth strut is attached to the second end of the second hoop arc.

27. The device of embodiment 26 wherein the first ends of the first and second hoop arcs are in closer proximity to each other than to the second ends of the first and second hoop arcs.

28. The device of embodiments 21-27 wherein the struts arc out from the guidewire when the device is in an expanded position.

29. The device of embodiments 21-27 further comprising a restraining element over the struts when the device is in a collapsed position.

30. The device of embodiment 29 wherein the restraining member is a delivery sleeve.

31. The device of embodiment 29 wherein the restraining member is a cardigan.

32. A method of providing embolic protection for an intravascular therapy at a region of interest, comprising the steps of:

(a) advancing a guide catheter to a point proximal the region of interest;

(b) advancing any of the devices of embodiments 1-31 through the guide catheter while in a collapsed configuration

(c) advancing the device through the region of interest;

(d) deploying the device by moving the device to an expanded configuration.

33. The method of embodiment 32 wherein steps (a) and (b) are performed simultaneously.

34. The method of embodiment 32 wherein the steps are performed in the order given.

35. The method of any of embodiments 32-34 wherein the device is a device of embodiments 1-28 and wherein the step of deploying the device involves moving an actuation member relative to the guidewire.

36. The method of embodiment 35 wherein the guidewire is held in place while the actuation member is moved.

37. The method of embodiment 35 wherein the actuation member is held while the guidewire is moved.

38. The method of any of embodiments 32-34 wherein the device is a device of embodiments 29-31 and wherein the step of deploying the device includes the step of removing the restraining member,

39. The method of any of embodiments 32-38 further comprising the step of performing a therapeutic procedure at the region of interest.

40. The method of embodiment 39 wherein the therapeutic procedure is an angioplasty procedure.

41. The method of any of embodiments 32-40 further comprising the step of collecting emboli in the filtering membrane.

42. The method of embodiment 41 further comprising the step of aspirating material from within the filtering membrane.

43. The method of any of embodiments 32-42, further comprising the step of withdrawing the device.

44. The method of claim 43 wherein the step of withdrawing the device includes the step of advancing a retrieval sheath over at least a proximal portion of the filtering membrane.

Merely for clarity, some Figures show only certain elements of the invention while not showing certain other elements. It will be understood that these elements may be combined as desired in practicing the invention.

The embodiments of this invention may be used with other devices. For example, the device may be delivered to the area of interest in a delivery sheath and collected by a retrieval sheath. Other therapy catheters such as stent delivery catheters and angioplasty catheters may be delivered over the device. Further, other components such as radiopaque bands and atraumatic distal tips may be used with embodiments of this invention. In short, the embodiments of this invention are distal protection devices and may be used with components and other devices typically used with such devices.

It should be understood that this disclosure is, in many respects, only illustrative. Changes may be made in details, particularly in matters of shape, size, and arrangement of steps without exceeding the scope of the invention. The scope of the invention is, of course, defined in the language in which the appended claims are expressed.

What is claimed is:

1. An intravascular distal protection device, comprising:
a guidewire having a proximal end and a distal end;
a filtering material disposed about the guidewire, the filtering material having a proximal edge defining a proximally facing opening when in an expanded position;
a hoop wire having a proximal end and a distal end and slidably attached to the proximal edge, the hoop wire wrapped in a helical coil about the guidewire in a first contracted position and defining a hoop at the proximal end of the filtering material in the expanded position; and
an actuation member attached to the hoop wire and extending at least substantially to the proximal end of the guidewire and configured to move the hoop wire between the first contracted position and the expanded position.

2. The device of claim 1, wherein the actuation member is attached to the distal end of the hoop wire.

3. The device of claim 1, wherein the actuation member is attached to the proximal end of the hoop wire.

4. The device of claim 1, wherein the helical coil includes two or more turns around the guidewire.

5. The device of claim 1, wherein the guidewire is hollow along at least a portion and wherein the actuation member is at least partially disposed within the guidewire.

6. The device of claim 1, wherein the proximal edge of the filtering material includes a hem disposed on the hoop wire.

7. The device of claim 1, wherein the proximal edge of the filtering material includes a plurality of attachment rings disposed on the hoop wire.

8. The device of claim 7, wherein the proximal edge of the filtering material includes one or more coils disposed between the attachment rings on the hoop wire.

9. The device of claim 1, wherein the filter has a length, and wherein in the first contracted position, the proximal end of the hoop wire is spaced apart from the distal end of the hoop wire a distance that is at least half the length of the filter.

10. The device of claim 1, wherein the distance is at least three-quarters the length of the filter.

11. The device of claim 1, further comprising a stop mechanism to connect the ends of the hoop when in the expanded position, the stop mechanism including a ring slidably disposed around the guidewire and connected to one of the hoop wire ends.

12. The device of claim 1 wherein the hoop wire is hollow, wherein the actuation member is disposed within the hoop wire and wherein tension in the actuation member brings the proximal and distal ends of the hoop wire together.

13. An intravascular distal protection device, comprising:
a hollow guidewire having a proximal end and a distal end;
a filtering material having a proximal edge defining a proximally facing opening when in an expanded position;
a hoop wire having a proximal end and a distal end and slidably attached to the proximal edge, the hoop wire having a reduced profile in a first contracted position and defining a hoop at the proximal end of the filtering material in the expanded position; and
an actuation member attached to the distal end of the hoop wire and configured to move the hoop wire between the first contracted position and the expanded position, the actuation member disposed within the guidewire.

14. The device of claim 13 wherein the hoop wire is wrapped about the actuation member in the contracted position.

15. The device of claim 13 wherein the filtering material is at least partially distal the distal end of the guidewire.

16. The device of claim 13, wherein the proximal edge of the filtering material includes a hem disposed on the hoop wire.

17. The device of claim 13, wherein the proximal edge of the filtering material includes a plurality of attachment rings disposed on the hoop wire.

18. The device of claim 17, wherein the proximal edge of the filtering material includes one or more coils disposed between the attachment rings on the hoop wire.

19. A method of using the device of claim 1, comprising the steps of:
positioning a delivery catheter proximal of an area of interest, the delivery catheter having an open distal end;
moving the device distally through the distal end of the delivery catheter and across the area of interest while the hoop wire is in the first contracted position; and
deploying the filtering material into the expanded position by moving the actuation member relative to the guidewire.

20. The method of claim 20, wherein the step of deploying the filtering material includes the step of pulling on the actuation member.
